(19) United States
(12) Patent Application Publication
(10) Pub. No.: US 2003/0158089 A1
(43) Pub. Date: Aug. 21, 2003

(54) Administrative agents via the SMVT transporter

(75) Inventors: Mark A. Gallop, Los Altos, CA (US); Kenneth C. Cundy, Redwood City, CA (US); Noa Zerangue, San Mateo, CA (US); Feng Xu, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: XenoPort, Inc., Santa Clara, CA

(21) Appl. No.: 10/350,893

(22) Filed: Jan. 23, 2003

Related U.S. Application Data
(60) Provisional application No. 60/351,808, filed on Jan. 24, 2002.

(51) Int. Cl. A61K 38/00; A61K 31/4188

(52) U.S. Cl. 514/2; 514/393

(57) ABSTRACT

Disclosed herein are conjugates comprising a therapeutic agent (e.g., a drug) which is linked to a conjugate moiety that is itself, or itself in combination with the agent, is a good substrate for the sodium dependent multi-vitamin transporter (SMVT). The conjugates have a molecular weight below 1500 daltons and exhibit increased uptake via SMVT through the cells lining the gastrointestinal lumen, and hence higher bioavailability, when administered orally compared to the therapeutic agent itself Also disclosed are methods of delivering agents that, as a result of linkage to a conjugate moiety, are good substrates of the SMVT transporter. Further disclosed are methods of screening conjugates or conjugate moieties, linked or linkable to a therapeutic agent, for capacity to be transported as substrates through the SMVT transporter.

### CROSS-REFERENCE TO RELATED APPLICATIONS

The present application derives priority from U.S. patent application Ser. No. 60/351,808 filed Jan. 24, 2002, incorporated by reference in its entirety for all purposes.

### BACKGROUND

Recent advances in the pharmaceutical industry have resulted in the formation of an increasing number of potential therapeutic agents. However, formulating certain compounds for effective oral delivery has proven difficult because of problems associated with poor uptake and high susceptibility to metabolic enzymes.

Natural transporter proteins are involved in the uptake of various molecules into and/or through cells. In general, two major transport systems exist: solute carrier-mediated systems and receptor mediated systems. Carrier-mediated systems use transport proteins that are anchored to the cell membrane, typically by a plurality of membrane-spanning loops and function by transporting their substrates via an energy-dependent flip-flop or other mechanism, exchange and other facilitative or equilibrative mechanisms. Carrier-mediated transport systems are involved in the active or non-active, facilitated uptake of many important nutrients, such as vitamins, sugars, and amino acids, from the gastrointestinal tract lumen into the blood. Separating the gastrointestinal tract lumen and the blood is an epithelial cell tissue, including a layer of enterocytes lining the gut lumen. The carrier systems result in transport of these nutrients into and through the enterocytes and across the epithelial cell tissue from lumen into blood (absorption). Other carrier-mediated transport systems have been shown to be involved in the transport of certain materials (e.g., toxins) from the blood, across the epithelial tissue to the lumen (secretion). Carrier-mediated transporters are also present in organs such as the liver and kidney, in which the proteins are involved in the excretion or re-absorption of circulating compounds.

Receptor-mediated transport systems differ from the carrier-mediated systems in that these systems usually utilize proteins that span the cell membrane only a single time. Furthermore, substrate binding triggers an invagination and encapsulation process that results in the formation of various transport vesicles to carry the substrate (and sometimes other molecules) into and through the cell. This process of membrane deformations that result in the internalization of certain substrates and their subsequent targeting to certain locations in the cytoplasm is generally referred to as endocytosis.

Polar or hydrophilic compounds typically exhibit poor passive diffusion across the intestinal wall/epithelia as there is a substantial energetic penalty for passage of such compounds across the lipid bilayers that constitute cellular membranes. Many nutrients that result from the digestion of ingested foodstuffs in animals, such as amino acids, di- and tripeptides, monosaccharides, nucleosides and water-soluble vitamins, are polar compounds whose uptake is essential to the viability of the animal. For these substances there exist specific mechanisms for active transport of the solute molecules across the intestinal epithelia. This transport is frequently energized by co-transport of ions down a concentration gradient.

The essential micronutrients pantothenate and biotin are two water-soluble vitamins for which a specific transport system has been identified in absorptive epithelial tissues, including intestine. A Na⁺-dependent vitamin transporter termed SMVT (sodium-dependent multivitamin transporter) has been recently cloned from rat, human and rabbit tissue (see Prasad et al, J. Biol. Chem. 1998, 273, 7501-7506; Wang et al, J. Biol. Chem. 1999, 274, 14875-14883; Chatterjee et al, Am. J. Physiol. 1999, 277, C605-C613; Prasad et al, Arch. Biochem. Biophys. 1999, 366, 95-106). The cDNA's of these genes code for highly homologous proteins of 634, 635 and 636 amino acids respectively (the human protein shows 84% identity, 89% similarity and 87% identity, 92% similarity to the rat and rabbit sequences respectively) with a predicted membrane topology of 12 transmembrane domains. SMVT also accepts the essential metabolite lipoate as a substrate and transporter affinities in the range of ˜1-20 μM have been measured for pantothenate, lipoate and biotin in a variety of cellular assays. Although the Na⁺-dependency of this transporter is unequivocal, there has been some controversy surrounding the exact stoichiometry of substrate: sodium ion co-substrate coupling, with proposals favoring both a Na⁺: substrate stoichiometry of 2:1 and 1:1 having been made. Recent studies of SMVT expression in Xenopus oocytes have demonstrated that, in the presence of sodium ions, substrate addition induces an electrogenic response consistent with a net flux of positive charge across the cellular membrane.

Transport of the vitamin substrates is thus energized by both the Na⁺ gradient as well as the potential difference that exists across the cell membrane (Prasad et al, Biochem. Biophys. Res. Commun. 2000, 270, 836-840; Prasad et al, Arch. Biochem. Biophys. 1999, 366, 95-106). The magnitudes of the maximal current response induced by either biotin, panthothenate or lipoate are roughly equivalent, indicating that each compound is approximately equally well transported by SMVT. Moreover, this assay offers more direct and meaningful assessment of substrate capability than competition type assays which simply measure the ability of a test compound to block the uptake of a labeled control substrate, and cannot distinguish compounds that are non-transported ligands for the transporter (i.e. inhibitors) from bona fide transporter substrates.

Competition type assays have previously been employed to examine the substrate specificity of SMVT in cells naturally expressing the transporter, as well as cells transfected with the gene encoding SMVT from human, rat and rabbit (see previous references, and Said et al, Am. J. Physiol. 1998, 275, C1365-C1371). Structural analogs of biotin that retain the free carboxylic acid moiety of the valeryl side chain (e.g., desthiobiotin, diaminobiotin) have been shown to interact with the transporter, while biotin analogs with a blocked carboxyl moiety (e.g., biocytin and biotin methyl ester) showed minimal interaction. This has led to the suggestion that the transporter favors anionic substrates and forms a key interaction with the carboxylate moiety shared by biotin, pantothenate and lipoate. Ramanathan et al, in Pharm. Res. 2001, 18, 950-956 and J. Controlled Release 2001, 77, 199-212, studied the cellular uptake and transcellular flux of biotinylated peptides derived from the HIV TAT sequence. In this work, high molecular weight conjugates of biotin coupled via the carboxylate moiety as an amide to either a 10 amino acid peptide (M.W.˜1,600 Da) alone, or in the context of a larger polyethylene glycol conjugate (molecular weight of about 29,000 Da), were shown to interact preferentially with cells expressing SMVT over non-transfected cells. Stein et al, in International Publication No. WO 02/062396, disclose high molecular weight (greater than 1,500 Da) polymer conjugates of therapeutic or diagnostic agents with one or more cell uptake promoters, which promoters include the biotinylated TAT peptides noted above. These polymers are said to have utility in the delivery of therapeutic or diagnostic agents from an initial bodily compartment to one or more target bodily compartments, including the oral delivery of agents to the target bodily compartment. While Stein et al note that the appendage of biotin as a targeting moiety to large peptides may enhance their intestinal absorption, they teach that the low capacity nature of SMVT makes this transporter susceptible to saturation, thereby limiting the dose of drug deliverable via this pathway. Conjugation of an agent to a polymer bearing multiple copies of this targeting moiety (i.e. the cellular uptake promoter) is proposed as a method to overcome this drawback.

### DISCLOSURE

The methods and compounds disclosed herein permit improved oral absorption of a pharmaceutical agent via its conversion to a conjugate derivative, which conjugate is a better substrate for the SMVT transporter expressed in the intestine of an animal, compared to the pharmaceutical agent itself. The conjugate has a molecular weight of less than 1500 Da. and comprises an agent, which agent is not a substrate, or is at least a poor substrate, for an SMVT transporter, linked to a conjugate moiety, via a cleavable linker, such that the conjugate is a substrate for, or is a better substrate for, an SMVT transporter. The conjugate has a Vₘₐₓ of at least 5% of biotin for the SMVT transporter. The conjugate has a greater Vₘₐₓ for SMVT than the Vₘₐₓ of the agent alone, i.e., without the conjugate moiety. Once the conjugate is taken up via the SMVT, the moiety is cleaved, thereby releasing the agent. The agent itself may be pharmacologically active, or a metabolite of the agent may be pharmacologically active. The data reported herein support the previously unappreciated finding that the SMVT transport pathway in the intestine provides a surprisingly high capacity uptake mechanism for conjugates of molecular weight below 1,500 Da.

Preferably, the conjugate has a Vₘₐₓ for the SMVT transporter of at least 10%, more preferably at least 20%, still more preferably at least 50%, and most preferably at least 100%, respectively, of the Vₘₐₓ of the substrate biotin for SMVT.

Although the conjugates and methods described herein are not limited to agents having any particular % Vₘₐₓ of the substrate biotin for SMVT, the conjugates and methods have greater utility as the % Vₘₐₓ of the agent (without the conjugate moiety) becomes lower, since agents with an already high % Vₘₐₓ may inherently exhibit sufficiently good uptake via SMVT. Thus, the conjugates and methods disclosed herein have particular utility when the pharmaceutical agent, without the conjugate moiety, has a Vₘₐₓ for the SMVT transporter of less than 5% of the Vₘₐₓ of substrate biotin for SMVT.

Also disclosed herein is a method of delivering an agent comprising orally administering the conjugate described above to a patient. The conjugate is formulated with a carrier for oral delivery. The carrier may comprise an immediate release formulation or a sustained release formulation.

Also disclosed herein is a method of making a pharmaceutical composition comprising forming a plurality of different conjugates, each conjugate comprising a single therapeutic agent linked to one of a plurality of different cleavable conjugate moieties. As mentioned above, the agent without any conjugate moiety, or an active metabolite of the agent, has a pharmacological activity. Each of the conjugates has a molecular weight of less than 1,500 Da. The plurality of conjugates are screened for SMVT transport. The screening is typically accomplished by contacting each of the conjugates with cells expressing the SMVT transporter and measuring conjugate uptake by the cells. Following screening, a conjugate having a Vₘₐₓ for the SMVT transporter of at least 5% of the Vₘₐₓ of substrate biotin for the SMVT transporter is selected, the selected conjugate having a greater Vₘₐₓ for the SMVT transporter than the agent without the selected conjugate moiety. The selected conjugate is then formulated as a pharmaceutical product, e.g., for testing, regulatory approval and eventual commercialization.

Further disclosed herein is a method of screening a conjugate for transport by an SMVT transporter. The method includes providing a conjugate comprising an agent cleavably linked to a conjugate moiety, the conjugate having a molecular weight of less than 1500 Da, contacting the conjugate with cell(s) expressing an SMVT transporter, and then determining whether the conjugate passes into and/or through the cell(s) by way of the transporter. Preferably, the cell(s) is transfected with DNA encoding the SMVT transporter. More preferably, the cell(s) is an oocyte injected with nucleic acid encoding the SMVT transporter.

Also disclosed herein are methods of making a pharmaceutical composition. Such methods include linking an agent to a conjugate moiety to form a conjugate wherein the conjugate is transported by the SMVT transporter with a Vₘₐₓ of at least 5% of the Vₘₐₓ of the substrate biotin. The conjugate is then formulated with a carrier as a pharmaceutical composition.

### DEFINITIONS

The phrase “specifically binds” when referring to a protein refers to a binding reaction which is determinative of the presence of the protein in the presence of a heterogeneous population of proteins and other biologics. Thus, under designated conditions, a specified ligand binds preferentially to a particular protein and does not bind in a significant amount to other proteins present in the sample. A molecule such as an antibody that specifically binds to a protein often has an association constant of at least 10⁶ M⁻¹ or 10⁷ M⁻¹, preferably 10⁸ M⁻¹ to 10⁹ M⁻¹, and more preferably, about 10¹⁰ M⁻¹ to 10¹¹ M⁻¹ or higher. However, some substrates of intestinal transporter proteins (such as SMVT) have much lower affinities (˜10³ M⁻¹) and yet the binding can still be shown to be specific.

A “transport protein” is a protein that has a direct or indirect role in transporting a molecule into and/or through a cell. The term includes, for example, membrane-bound proteins that recognize a substrate and effect its entry into, or exit from a cell by a carrier-mediated transporter or by receptor-mediated transport. These proteins are sometimes referred to as transporter proteins. The term also includes intracellularly expressed proteins that participate in trafficking of substrates through or out of a cell. The term also includes proteins or glycoproteins exposed on the surface of a cell that do not directly transport a substrate but bind to the substrate holding it in proximity to a receptor or transporter protein that effects entry of the substrate into or through the cell. Examples of carrier proteins include: the intestinal and liver bile acid transporters, dipeptide transporters (e.g. PEPT1 and PEPT2), oligopeptide transporters, multivitamin transporters (e.g. SMVT), simple sugar transporters (e.g., SGLT1), phosphate transporters, monocarboxylic acid transporters, transporters comprising P-glycoproteins, organic anion transporters (OATP), and organic cation transporters. Examples of receptor-mediated transport proteins include: viral receptors, immunoglobulin receptors, bacterial toxin receptors, plant lectin receptors, bacterial adhesion receptors, vitamin B12 transporters and cytokine growth factor receptors. “SMVT” and “SMVT transporter” both refer to the sodium-dependent multivitamin transporter (SLC5A6). Genes encoding this transporter have been cloned from rat, human and rabbit tissue (see Prasad et al, J. Biol. Chem. 1998, 273, 7501-7506; Wang et al, J. Biol. Chem. 1999, 274, 14875-14883; Chatterjee et al, Am. J. Physiol. 1999, 277, C605-C613; Prasad et al, Arch. Biochem. Biophys. 1999, 366, 95-106). SMVT is expressed in intestinal, brain, liver, kidney, skeletal muscle, lung, pancreas and placental tissue. Endogenous substrates for SMVT are the micronutrients pantothenate, biotin and lipoate, each of which has binding affinities for the transporter in the range of 1-20 μM.

A “substrate” of a transport protein is a compound whose uptake into or passage through a cell is facilitated by the transport protein.

The term “ligand” of a transport protein includes substrates and other compounds that bind to the transport protein without being taken up or transported through a cell. Some ligands by binding to the transport protein inhibit or antagonize uptake of the substrate or passage of substrate through a cell by the transport protein. Some ligands by binding to the transport protein promote or agonize uptake or passage of the compound by the transport protein or another transport protein. For example, binding of a ligand to one transport protein can promote uptake of a substrate by a second transport protein in proximity with the first transport protein.

The term “agent” is used to describe a compound that has or may have a pharmacological activity, or may be converted to a compound that has pharmacological activity in the body. Agents include compounds that are known drugs, compounds for which pharmacological activity has been identified but which are undergoing further therapeutic evaluation, and compounds that are members of collections and libraries that are to be screened for a pharmacological activity.

An agent is “orally active” if it can exert a pharmacological activity when administered via an oral route.

A “conjugate” comprises a pharmaceutical agent, which agent is not a substrate, or is at least a poor substrate, for the SMVT transporter, linked to a conjugate moiety such that the conjugate is a substrate for, or is a better substrate for, the SMVT transporter.

A “conjugate moiety” refers to a chemical moiety which can be linked to an agent to form a conjugate that is a substrate for the SMVT transporter. The conjugate moiety facilitates therapeutic use of the agent by promoting uptake of the agent via the SMVT transporter. A conjugate moiety can itself be a substrate for the transporter (e.g., pantothenic acid as a substrate for the SMVT transporter) or can become a substrate when covalently linked to an agent. Thus, a conjugate formed from an agent and a conjugate moiety has higher uptake via SMVT than the agent alone and the conjugate may have higher uptake via SMVT than the conjugate moiety alone. In certain cases the conjugate moiety may be selected to be cleavable in vivo, such that once the conjugate is taken up by SMVT, the linker is cleaved and the agent is released.

“Pharmacological activity” means that an agent at least exhibits an activity in a screening system that indicates that the agent is or may be useful in the prophylaxis or treatment of a disease. The screening system can be in vitro, cellular, animal or human. Agents can be described as having pharmacological activity notwithstanding that further testing may be required to establish actual prophylactic or therapeutic utility in treatment of a disease.

“Vₘₐₓ” and “K_{M}” of a compound for a transporter (e.g., the SMVT transporter) are defined in accordance with convention. Vₘₐₓ is the number of molecules of compound transported per unit time at saturating concentration of the compound. K_{M} is the concentration of the compound at which the compound is transported at half of Vₘₐₓ. In general, a high value Of Vₘₐₓ is desirable for a substrate of a transporter. A low value of K_{M} is desirable for transport of low concentrations of a compound, and a high value of K_{M} is desirable for transport of high concentrations of a compound. Vₘₐₓ is affected both by the intrinsic turnover rate of a transporter (molecules/transporter protein) and transporter density in plasma membrane that depends on expression level. For these reasons, the intrinsic capacity of a compound to be transported by a particular transporter is usually expressed as the ratio (or percent) of the Vₘₐₓ of the compound/Vₘₐₓ of a control compound known to be a substrate for the transporter. Biotin is known to be a good substrate for SMVT and hence is used herein as the SMVT control compound. When biotin is added to voltage-clamped Xenopus oocytes expressing human or rat SMVT, a maximal sodium-dependent electrogenic response of typically 50-100 nA is observed, corresponding to a Vₘₐₓ of 25-50 pmol/oocyte/hour and a K_{M} in the range 5-20 μM (Prasad et al, Biochem. Biophys. Res. Commun. 2000, 270, 836-840; Wang et al, J. Biol. Chem. 1999, 274, 14875-14883). In order to determine how good a test compound is as a substrate for SMVT (i.e., how well a test compound is transported by SMVT), the Vₘₐₓ of the test compound is expressed as a percentage of the Vₘₐₓ of biotin, reported as the percentage of the maximum biotin current response induced by the test compound (at a screening concentration of 500 μM), as described in more detail in Example 4 herein.

“Transporter expression” refers to the presence and/or degree to which a particular transporter (e.g., the SMVT transporter) is found in the cells of a particular tissue (e.g., the enterocytes lining the gastrointestinal tract lumen).

A transporter is expressed in a particular tissue, e.g., the jejunum, when expression can be detected by mRNA analysis, protein analysis, antibody histochemistry, or functional transport assays. Typically, detectable mRNA expression is at a level of at least 0.01% of that of beta actin in the same tissue. Preferred transporters exhibit levels of expression in the desired tissue of at least 0.1, or 1 or 10% of that of beta actin. Conversely a transporter is not expressed in a particular tissue if expression is not detectable above experimental error by any of the above techniques. Thus, transporters that are not expressed in particular tissue exhibit express levels less than 0.1% of beta actin, and usually less than 0.01% of beta actin.

“Sustained release” refers to release of a therapeutic or prophylactic amount of the agent, conjugate or an active metabolite thereof into the systemic blood circulation over a prolonged period of time relative to that achieved by oral administration of a conventional immediate release formulation of the agent, conjugate or active metabolite. Typically, oral sustained release formulations release their active ingredients over a period of at least 3 hours, more typically over periods of 6-24 hours.

### DETAILED DESCRIPTION

1. Introduction

Disclosed herein are methods of screening agents, conjugates or conjugate moieties, linked or linkable to agents, for capacity to be transported as substrates through the SMVT transporter. Also disclosed are methods of treatment involving oral delivery of agents that, as a result of linkage to a conjugate moiety, are substrates of the SMVT transporter. SMVT is expressed in the human intestine, particularly the stomach, jejunum, ileum, the ileo-caecal valve, the cecum and the ascending colon.

SMVT can be cloned from rat, human and rabbit tissue following the methods described in Prasad et al, J. Biol. Chem. 1998, 273, 7501-7506; Wang et al, J. Biol. Chem. 1999, 274, 14875-14883; Chatterjee et al, Am. J. Physiol. 1999, 277, C605-C613; Prasad et al, Arch. Biochem. Biophys. 1999, 366, 95-106, the disclosures of which are incorporated by reference in their entirety. The cDNA's of these genes code for highly homologous proteins of 634, 635 and 636 amino acids respectively (the human protein shows 84% identity, 89% similarity and 87% identity, 92% similarity to the rat and rabbit sequences respectively) with a predicted membrane topology of 12 transmembrane domains.

2. Methods of Identifying Conjugates or Conjugate Moieties that are Substrates of the SMVT Transporter

Conjugates (i.e., the compound formed upon linking the conjugate moiety to the agent) can be screened directly for their capacity to act as substrates of the SMVT transporter. Alternatively, conjugate moieties can be screened as substrates, and the conjugate moieties linked to agents having known or suspected pharmacological activity. In such methods, the conjugate moieties can be linked to an agent or other molecule as a conjugate prior to screening. If another molecule is used, the molecule is sometimes chosen to resemble the chemical structure of an agent ultimately intended to be linked to the conjugate moiety for pharmaceutical use. The screening is typically performed on cells expressing the SMVT transporter. In some methods, the cells are transfected with DNA encoding the SMVT transporter. In other methods, cells naturally expressing the SMVT transporter are used. In some methods, SMVT is the only transporter expressed. In other methods, cells express SMVT in combination with other transporters. In still other methods, conjugates or conjugate moieties are screened on different cells expressing different transporters. For example, conjugates or conjugate moieties can be screened on cells expressing SMVT. Methods of screening conjugates or conjugate moieties for passage through cells expressing transporters are described in International Patent Application WO 01/20331, the disclosures of which are incorporated herein by reference.

Substrate transport by SMVT has an obligatory dependence on Na⁺ ions as cosubstrate. When expressed in Xenopus oocytes, SMVT responds electrogenically (induction of an inward current) upon addition of a substrate, with the magnitude of the response being directly proportional to the rate of substrate transport. As described in Example 4 herein, electrophysiological measurements provide a convenient method for evaluating the transport properties of SMVT substrates.

LC-MS assay methods are also valuable approaches to detecting transport of substrates into SMVT transfected cells. Test compounds are incubated with transporter-expressing cells for some period of time, washed, lysed and the cellular contents analyzed by LC-MS to detect transported substrates. Non-expressing cells provide a control for transporter-independent uptake (e.g., via passive diffusion).

Internalization (within a cell) of a compound evidencing passage of the compound through the plasma membrane via SMVT can also be detected by detecting a signal from within an SMVT-expressing cell from any of a variety of reporters. The reporter can be a label such as a fluorophore, a chromophore or a radioisotope. Confocal imaging can also be used to detect internalization of a label as it provides sufficient spatial resolution to distinguish between fluorescence on a cell surface and fluorescence within a cell; alternatively, confocal imaging can be used to track the movement of compounds over time. In another approach, internalization of a compound is detected using a reporter that is a substrate for an enzyme expressed within a cell. Once the complex is internalized, the substrate is metabolized by the enzyme and generates an optical signal or radioactive decay that is indicative of uptake. Light emission can be monitored by commercial photomultiplier tube (PMT) based instruments or by charged coupled device (CCD) based imaging systems.

In some methods, multiple conjugates or conjugate moieties are screened simultaneously and the identity of each conjugate or conjugate moiety is tracked using tags linked to the conjugates or conjugate moieties. In some methods, a preliminary step is performed to determine binding of a conjugate or conjugate moiety to SMVT. Although not all conjugates or conjugate moieties that bind SMVT are substrates of the transporter, observation of binding is an indication that allows one to reduce the number of candidate substrates from an initial repertoire. In some methods, substrate capacity of a conjugate or conjugate moiety is tested in comparison with a reference substrate of SMVT. Biotin is used as a reference substrate for SMVT transport studies. The comparison can either be performed in separate parallel assays in which a conjugate or conjugate moiety under test and biotin are compared for uptake on separate samples of the same cells. Alternatively, the comparison can be performed in a competition format in which a conjugate or conjugate moiety under test and biotin are applied to the same cells. Typically, the conjugate or conjugate moiety and biotin are differentially labeled in such assays.

In such comparative assays, the Vₘₐₓ of a conjugate moiety, or conjugate comprising an agent and conjugate moiety tested can be compared with that of biotin. If an agent, conjugate moiety or conjugate has a Vₘₐₓ of at least 5%, preferably at least 10%, more preferably at least 20%, and most preferably at least 50% of the Vₘₐₓ of biotin for the SMVT transporter, then the agent, conjugate moiety or conjugate can be considered to be a substrate for SMVT. In general, the higher the Vₘₐₓ of the conjugate moiety or conjugate relative to that of biotin the better. Therefore, conjugate moieties or conjugates having Vₘₐₓ's of at least 25%, 50%, 100% or 150% of the Vₘₐₓ of biotin for SMVT are screened in some methods.

As mentioned earlier, the conjugates and methods described herein are not limited to agents having any particular % Vₘₐₓ of the substrate biotin for SMVT. However, the conjugates and methods have greater utility as the % Vₘₐₓ of the agent (without the conjugate moiety) becomes lower, since agents with an already high % Vₘₐₓ may inherently exhibit sufficiently good uptake via SMVT. Thus, the conjugates and methods disclosed herein have particular utility when the agent, without the conjugate moiety, has a Vₘₐₓ for the SMVT transporter of less than 5% of the Vₘₐₓ of substrate biotin for SMVT, and more preferably less than 1% of the Vₘₐₓ of substrate biotin for SMVT. Methods of measuring the Vₘₐₓ of an agent as substrate for SMVT are substantially the same as the methods outlined earlier herein with respect to measuring the Vₘₐₓ of conjugates and conjugate moieties.

Example 5 herein examines the electrophysiological response of Xenopus oocytes transfected with the human SMVT transporter to a highly purified preparation of the biotinylated TAT peptide sequence, N-acetyl-D-Lys(ε-biotin)-D-Arg-D-Arg-D-Arg-D-Gln-D-Arg-D-Arg-D-Lys-D-Lys-D-Arg-NH₂, at a concentration of 20 μM, both in the presence and absence of 100 mM Na⁺ ions (in Na⁺-free solutions K⁺ or choline was used in place of Na⁺ ions). No specific sodium-dependent current was induced by this peptide, indicating that this compound is not a substrate for SMVT, contrary to the conclusions of Ramanathan et al and Stein et al (vide supra).

3. Agents, Conjugates and Conjugate Moieties to be Screened

Compounds constituting agents, conjugates or conjugate moieties to be screened can be naturally occurring or synthetic molecules. Natural sources include sources such as, e.g., marine microorganisms, algae, plants, and fungi. Alternatively, compounds to be screened can be from combinatorial libraries of agents, including peptides or small molecules, or from existing repertories of chemical compounds synthesized in industry, e.g., by the chemical, pharmaceutical, environmental, agricultural, marine, cosmeceutical, drug, and biotechnological industries. Compounds can include, e.g., pharmaceuticals, therapeutics, environmental, agricultural, or industrial agents, pollutants, cosmeceuticals, drugs, organic compounds, lipids, glucocorticoids, antibiotics, peptides, sugars, carbohydrates, and chimeric molecules. Preferred conjugates contain a free carboxylic acid moiety.

4. Linkage of Agents to Conjugate Moieties

Conjugate moieties that are substrates for SMVT can be attached to or incorporated into agents having pharmacological activity by a variety of means. Conjugates can be prepared by either direct conjugation of an agent to a conjugate moiety, or by covalently coupling a difunctionalized linker precursor with an agent to a conjugate moiety. The linker precursor is selected to contain at least one reactive functionality that is complementary to at least one reactive functionality on the agent and at least one reactive functionality on the conjugate moiety. Such complementary reactive groups are well known in the art as illustrated below:

### COMPLEMENTARY BINDING CHEMISTRIES

| First Reactive Group | Second Reactive Group | Linkage |
|---|---|---|
| hydroxyl | carboxylic acid | ester |
| hydroxyl | haloformate | carbonate |
| thiol | carboxylic acid | thioester |
| thiol | haloformate | thiocarbonate |
| amine | carboxylic acid | amide |
| hydroxyl | isocyanate | carbamate |
| amine | haloformate | carbamate |
| amine | isocyanate | urea |
| amine | acyloxychloroformate | acyloxycarbamate |
| carboxylic acid | chloroalkylcarbamate | acyloxycarbamate |
| carboxylic acid | carboxylic acid | anhydride |
| hydroxyl | phosphorus acid | phosphonate or |
| | | phosphate ester |
| amine | phosphorus acid | phosphoramidate |

In addition to the complementary chemistry of the functional groups on the linker to both the agent and conjugate moiety, the linker is selected to be cleavable in vivo, such that once the conjugate is taken up by SMVT, the linker is cleaved and the agent is released. Cleavable linkers are well known in the art and are selected such that at least one of the covalent bonds of the linker that attaches the agent to the conjugate moiety can be broken in vivo thereby providing for the agent or active metabolite thereof to be available to the systemic blood circulation. The linker is selected such that the reactions required to break the cleavable covalent bond are favored at the physiological site in vivo which permits agent (or active metabolite thereof) release into the systemic blood circulation or other tissue.

The selection of suitable cleavable linkers to provide effective concentrations of the agent or active metabolite thereof for release into blood or tissue can be evaluated using endogenous enzymes in standard in vitro assays to provide a correlation to in vivo cleavage of the agent or active metabolite thereof from the conjugate, as is well known in the art and described further in Example 6 herein. It is recognized that the exact cleavage mechanism employed is not critical to the methods of this invention provided, of course, that the conjugate cleaves in vivo in some form to provide for the agent or active metabolite thereof for release into the systemic blood circulation or other tissue.

Examples of cleavable linkers suitable for use as described above include peptides with protease cleavage sites (see, e.g., U.S. Pat. No. 5,382,513). Other exemplary linkers that can be used are also described in International Patent Application WO 02/44324; European Patent Application 188,256; U.S. Pat. Nos. 4,671,958; 4,659,839; 4,414,148; 4,669,784; 4,680,338, 4,569,789 and 4,589,071, each of which is incorporated by reference in its entirety for all purposes.

The ability of the SMVT transport mechanism to mediate intestinal absorption of pharmaceutically useful quantities of low molecular weight substrates (i.e., less than 1,500 Da) is probed in the dose-escalation study reported in Example 7 below. Biotin and pantothenate, both natural substrates for this transporter, are administered by oral gavage to rats over the dose range of 10 mg/kg to 200 mg/kg. Both the maximal plasma concentration (Cₘₐₓ) and the area under the plasma concentration versus time curve (AUC) for these two molecules increased with dose, each attaining a maximal plasma concentration of about 4 μg/mL despite extensive systemic metabolic clearance. Thus in contrast to previous suggestions that delivery of drugs via the SMVT transporter is limited by the low capacity of this absorption pathway, the data reported herein support the oral delivery of useful doses of small molecule substrates of SMVT.

There are many existing drugs for which uptake through the intestine can be improved. Drugs suitable for conversion to conjugates that are capable of uptake from the intestine typically contain one or more of the following functional groups to which a moiety may be conjugated: primary or secondary amino groups, hydroxyl groups, carboxylic acid groups, phosphonic acid groups, or phosphoric acid groups. Examples of drugs containing carboxyl groups include, for instance, angiotensin-converting enzyme inhibitors, β-lactam antibiotics, non-steroidal antiinflammatory agents, prostaglandins and quinolone antibiotics. Examples of drugs containing amine groups include, for example, β-receptor blockers. Examples of drugs containing hydroxy groups include steroidal hormones, tranquilizers, neuroleptics, cytostatic or cytotoxic anticancer agents, macrolide antibiotics, antiviral agents, antifungal agents, protease inhibitors, glucocorticoids, narcotic agonists and antagonists, bronchodilators, anticoagulants and antihypocholesteremic agents. Representative drugs containing phosphonic or phospohoric acid moieties include bisphosphonate anti-osteoporosis agents, and antiviral or anticancer nucleoside derivatives.

The present invention has particular utility in the modification of those drugs (i.e., modified into conjugates) that exhibit poor gastrointestinal absorption. Thus the present invention is useful in the formation of prodrug conjugates of those drugs exhibiting oral bioavailabilities of less that 75%, preferably less that 50%, and most preferably less than 25%.

The oral absorption of a conjugate of the drug gabapentin in rats and primates is reported in Examples 8 and 9 respectively. Gabapentin, when administered orally, is known to have low bioavailability. Orally administering the gabapentin conjugate of Examples 8 and 9 achieved gabapentin bioavailabilities exceeding 50% in both species, confirming that this conjugate, a good SMVT substrate, is well absorbed in rats and monkeys after oral dosing.

6. Pharmaceutical Compositions and Methods of Treatment

The conjugates that are substrates for SMVT can be incorporated into pharmaceutical compositions. Usually, although not necessarily, such pharmaceutical compositions are designed for oral administration. Oral administration of such compositions results in uptake of the conjugate through the intestine via the SMVT transporter and entry into the systemic circulation. Note that it is not necessary that this intestinal uptake be exclusively mediated through the SMVT pathway (i.e., other active or passive transport pathways may also contribute to absorption of the conjugate). The pharmaceutical composition can thus be efficiently delivered to a wide range of tissues in the body.

The conjugates are combined with pharmaceutically-acceptable, non-toxic carrier(s) which are commonly used to formulate pharmaceutical compositions for animal or human oral administration. The carrier is selected so as not to adversely affect the biological activity of the combination. Examples of such carriers are distilled water, buffered water, physiological saline, PBS, Ringer's solution, dextrose solution, and Hank's solution. In addition, the pharmaceutical composition or formulation can also include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents, detergents and the like (see, e.g., “Remington's Pharmaceutical Sciences”, Mace Publishing Company, Philadelphia, Pa., 17th ed. (1985); for a brief review of methods for drug delivery, see, Langer, Science 249:1527-1533 (1990); each of these references is incorporated by reference in its entirety).

Pharnaceutical compositions for oral administration can be in the form of e.g., tablets, pills, powders, lozenges, sachets, cachets, elixirs, suspensions, emulsions, solutions, or syrups. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methylcellulose. Preserving agents such as methyl- and propylhydroxy-benzoates; sweetening agents; and flavoring agents can also be included. Depending on the formulation, compositions can provide quick, sustained or delayed release of the active ingredient after administration to the patient. The oral dosage forms used to deliver the conjugates may be formed, coated or otherwise compounded to provide a dosage form affording the advantage of prolonged action. For example, the oral dosage form can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of known polymeric acids and mixtures of polymeric acids with such materials as shellac, cetyl alcohol, and cellulose acetate.

For preparing solid compositions such as tablets, the principal active ingredient is mixed with a pharmaceutical excipient to form a solid preformulation composition containing a homogeneous mixture of a conjugate. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 mg to about 2 g of the active agent.

The conjugates can be administered for prophylactic and/or therapeutic treatments. A therapeutic amount is an amount sufficient to remedy a disease state or symptoms, or otherwise prevent, hinder, retard, or reverse the progression of disease or any other undesirable symptoms in any way whatsoever. In prophylactic applications, compositions are administered to a patient susceptible to or otherwise at risk of a particular disease, condition or infection. Hence, a “prophylactically effective amount” is an amount sufficient to prevent, hinder or retard a disease state or its symptoms. In either instance, the precise amount of conjugate contained in the composition depends on the patient's state of health and weight.

An appropriate dosage of the conjugate is readily determined according to any one of several well-established protocols. For example, animal studies (e.g., mice, rats) are commonly used to determine the maximal tolerable dose of the bioactive agent per kilogram of weight. In general, at least one of the animal species tested is mammalian. The results from the animal studies can be extrapolated to determine doses for use in other species, such as humans for example.

The components of pharmaceutical compositions are preferably of high purity and are substantially free of potentially harmful contaminants (e.g., at least National Food (NF) grade, generally at least analytical grade, and more typically at least pharmaceutical grade). To the extent that a given conjugate must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions. Compositions for oral administration need not be sterile or substantially isotonic but are usually made under GMP conditions.

### EXAMPLES

The following examples describe in detail preparation of specific compounds and compositions and specific assays for using the compounds and compositions. These examples should be viewed as illustrating specific examples of the invention, but should not be viewed as defining the scope of the invention.

In the examples below, the following abbreviations have the following meanings. If an abbreviation is not defined, it has its generally accepted meaning.

| | | |
|---|---|---|
| Atm | = | atmosphere |
| Boc | = | tert-butyloxycarbonyl |
| Cbz | = | carbobenzyloxy |
| CPM | = | counts per minute |
| DCC | = | dicyclohexylcarbodiimide |
| DMAP | = | 4-N,N-dimethylaminopyridine |
| DMEM | = | Dulbecco's minimun eagle medium |
| DMF | = | N,N-dimethylformamide |
| DMSO | = | dimethylsulfoxide |
| Fmoc | = | 9-fluorenylmethyloxycarbonyl |
| g | = | gram |
| h | = | hour |
| HBSS | = | Hank's buffered saline solution |
| L | = | liter |
| LC/MS | = | liquid chromatography/mass spectroscopy |
| M | = | molar |
| min | = | minute |
| mL | = | milliliter |
| mmol | = | millimoles |
| NHS | = | N-hydroxysuccinimide |
| PBS | = | phosphate buffered saline |
| THF | = | tetrahydrofuran |
| TFA | = | trifluoroacetic acid |
| TMS | = | trimethylsilyl |
| μl | = | microliter |
| μM | = | micromolar |
| v/v | = | volume to volume |

### EXAMPLE 1

### 1-{[(α-Pivaloyloxymethoxy)carbonyl]aminomethyl}-1-Cyclohexane Acetic Acid (1)

### Step A: Chloromethyl p-Nitrophenyl Carbonate (2)

p-Nitrophenol (100 g, 0.72 moles) was dissolved in anhydrous tetrahydrofuran (3 L) and stirred vigorously. To this solution was added chloromethyl chloroformate (70 mL, 0.79 moles) at room temperature followed by triethylamine (110 mL). After stirring for 1 hour, the reaction mixture was filtered and the filtrate was concentrated and then diluted with ethyl acetate (1 L). The organic solution was washed with 10% potassium carbonate (3×500 mL) and 1 N HCl (2×300 mL), brine (2×300 mL) and dried over anhydrous sodium sulfate. Removal of the solvent gave 157 g (95%) of the title compound (2) as a solid. The compound was unstable to LC-MS. ¹H NMR (CDCl₃, 400 MHz): 5.86 (s, 2H), 7.44 (d, J=9 Hz, 2H), 8.33 (d, J=9 Hz, 2H).

### Step B: Iodomethyl p-Nitrophenyl Carbonate (3)

Chloromethylp-nitrophenyl carbonate (2) (100 g, 0.43 moles), sodium iodide (228 g, 1.30 moles) and 50 g of dried molecular sieves (4 Å) were added to 2 L of acetone under nitrogen with mechanical stirring. The resulting mixture was stirred at 40° C. for 5 hours (monitored by ¹H NMR). Upon completion, the solid materials were removed by filtration and the solvent was removed under reduced pressure. The residue was redissolved in dichloromethane (1 L) and washed twice with saturated aqueous sodium carbonate (300 mL) followed by water (300 mL). The organic layer was separated and dried over anhydrous sodium sulfate. Removal of solvent gave 123.6 g (89%) of the title compound (3) as a solid upon standing. The compound was found to be unstable to LC-MS. ¹H NMR (CDCl₃, 400 MHz): 6.06 (s, 2H), 7.42 (d, J=9 Hz, 2H), 8.30 (d, J=9 Hz, 2H). ¹³C NMR (CDCl₃, 100 MHz): 155.1, 151.0, 146.0, 125.8, 125.7, 121.9, 33.5.

### Step C: Silver Pivalate (4)

Pivalic acid (50 g, 0.49 moles) was dissolved in acetonitrile (1.3 L) followed by addition of silver oxide (70 g, 0.29 moles) with vigorous stirring. Then, 660 mL of water was added under nitrogen. The resulting suspension was stirred at 70° C. in dark for 1 hour. After filtration through a pad of Celite, removal of the solvent gave 86 g (82%) of the title compound (4) as a pale white solid, which was used in the next reaction without further purification.

Other silver salts described in this application are prepared following similar procedures.

### Step D: p-Nitrophenyl Pivaloyloxymethyl Carbonate (5)

To a solution of iodomethylp-nitrophenyl carbonate (3) (62 g, 0.19 moles) in anhydrous toluene (1 L) was added silver pivalate (80 g, 0.38 moles). After stirring at 55° C. under nitrogen for 3 h, the reaction mixture was allowed to cool to room temperature and filtered through a pad of Celite. The filtrate was washed with 10% potassium carbonate (500 mL). Removal of the solvent yielded 43 g (75%) of the title compound (5) as a yellow oil. ¹H NMR (CDCl₃, 400 MHz): 1.25 (s, 9H), 5.88 (s, 2H), 7.40 (d, J=9 Hz, 2H), 8.29 (d, J=9 Hz, 2H). ¹³C NMR (CDCl₃, 100 MHz): 177.0, 155.3, 151.6, 145.8, 125.6, 121.9, 83.1, 39.1, 27.0.

### Step E: 1-{[(α-Pivaloyloxymethoxy)carbonyl]aminomethyl}-1-Cyclohexane Acetic Acid (1)

Gabapentin free base (24 g, 0.14 moles) was slurried in anhydrous dichloromethane (100 mL) and then treated with chlorotrimethylsilane (18.6 mL, 0.28 moles) and triethylamine (10 mL, 0.15 moles), respectively. The resulting suspension was warmed with stirring until complete dissolution of any solid was achieved. The above gabapentin solution was added via an equalizing addition funnel to a gently refluxed and mechanically stirred solution of p-nitrophenyl pivaloyloxymethyl carbonate (5) (20 g, 67 mmol) and triethylamine (10 mL, 0.15 moles) in dichloromethane (100 mL) under nitrogen. The resulting yellow solution was stirred for 1.5 hours. Upon completion (monitored by ninhydrin stain), the mixture was filtered and the filtrate was concentrated. The residue was dissolved in ethyl acetate (500 mL) and washed with 1N HCl (3×100 mL), brine (2×100 mL) and dried over anhydrous sodium sulfate. After removing the solvent, the crude product was dissolved in ethanol (300 mL) and then 1 g of 5% Pd/C was added. The resulting mixture was shaken under 50 psi hydrogen atmosphere for 15 minutes and then filtered through a pad of Celite. After concentration, the residue was dissolved in ethyl acetate, washed with 5% H₂SO₄ and dried over anhydrous sodium sulfate. After removing the solvent under reduced pressure, the residue was purified by chromatography on silica gel (4:1 hexanes:ethylacetate) to afford 15 g (68%) of the title compound (1) as a solid. M.p.: 79-81° C.; ¹H NMR (CDCl₃, 400 MHz): 1.21 (s, 9H), 1.3-1.5 (m, 10H), 2.32 (s, 2H), 3.26 (s, 2H), 5.33 (m, 1H), 5.73 (s, 2H). ¹³C NMR (CDCl₃, 400 MHz): 21.7, 26.2, 27.3, 34.3, 38.2, 39.2, 80.6, 155.9, 176.8, 178.0. MS (ESI) m/z 328.36 (M−H)³¹ , 330.32 (M+H)⁺, 352.33 (M+Na)⁺.

### EXAMPLE 2

### 1-{[(α-Isobutanoyloxyethoxy)carbonyl]aminomethyl}-1-Cyclohexane Acetic Acid (6)

### Step A: 1-Iodoethyl-p-Nitrophenyl Carbonate (7)

A mixture of 1-chloroethyl-p-nitrophenyl carbonate (2.5 g, 10 mmol) and Nal (3.0 g, 20 mmol) in dry acetone was stirred for 3 hours at 40° C. After filtration, the filtrate was concentrated under reduced pressure to afford 2.4 g (72%) of the title compound (7), which was used in the next reaction without further purification.

Step B: α-Isobutanoyloxyethoxy-p-Nitrophenyl Carbonate (8)

A mixture of 1-iodoethyl-p-nitrophenyl carbonate (7) (1.5 g, 4.5 mmol) and silver isobutyrate (1.3 g, 6.7 mmol) in toluene (40 mL) was stirred at 90° C. in an oil bath for 24 h. The reaction mixture was filtered and the filtrate was concentrated under reduced pressure. Chromatography of the resulting residue on silica gel, (20% CH₂Cl₂/hexanes and then 40% CH₂Cl₂/hexanes), gave 0.46 g (36%) of the title compound (8).

### Step C: 1-{[(α-Isobutanoyloxyethoxy)carbonyl]aminomethyl}-1-Cyclohexane Acetic Acid (6)

To a mixture containing gabapentin (530 mg, 3.1 mmol) and triethylamine (0.89 mL, 6.4 mmol) in dichloromethane (30 mL) was added trimethylchlorosilane (0.83 mL, 6.4 mmol) and the resulting mixture was stirred until a clear solution was formed. To this solution was added a solution of α-isobutanoyloxyethyl-p-nitrophenyl carbonate (8) (0.46 g, 1.6 mmol) in dichloromethane (10 mL) and the resulting mixture was stirred for 30 min. The reaction mixture was washed with 10% citric acid (20 mL) and the organic phase was separated. The aqueous layer was further extracted with ether (3×10 mL) and the combined organic phases were dried over MgSO₄, then concentrated in vacuo. The resulting residue was purified by reverse phase preparative HPLC (acetonitrile, water 0.1 % formic acid) to afford 70 mg (21%) of the title compound (6). ¹H NMR (CD₃OD, 400 MHz): 1.12 (d, J=7.2 Hz, 3H), 1.14 (d, J=7.2 Hz, 3H), 1.32-1.58 (m, 10H), 1.44 (d, J=5.6 Hz, 3H), 2.28 (s, 2H), 2.56 (m, 1H), 3.25 (m, 2H), 6.73 (q, J=5.6 Hz, 1H). MS (ESI) m/z 330.30 (M+H)⁺.

### EXAMPLE 3

### 1-{[(α-Propanoyloxyisobutoxy)carbonyl]aminomethyl}-1-Cyclohexane Acetic Acid (9)

### Step A: 1-Iodo-2-Methylpropyl-p-Nitrophenyl Carbonate (10)

A mixture of 1-chloro-2-methylpropyl-p-nitrophenyl carbonate (1.0 g, 4 mmol) and NaI (1.2 g, 8 mmol) in dry acetone was stirred for 3 hours at 40° C. After filtration, the filtrate was concentrated under reduced pressure to afford 510 mg (35%) of the title compound (10), which was used in the next reaction without further purification.

### Step B: α-Propanoyloxyisobutoxy-p-Nitrophenyl Carbonate (11)

A mixture of 1-iodo-2-methylpropyl -p-nitrophenyl carbonate (10) (0.51 g, 1.4 mmol) and silver propionate (0.54 g, 3 mmol) in toluene (20 mL) was stirred at 50° C. for 24 hours. The reaction mixture was filtered to remove solids and the filtrate concentrated under reduced pressure. Chromatography of the resulting residue on silica gel, (20% CH₂Cl₂/hexanes and then 40% CH₂Cl₂/hexanes), gave 0.39 g (89%) of the title compound (11).

### Step C: 1-{[(α-Propanoyloxyisobutoxy)carbonyl]aminomethyl}-1-Cyclohexane Acetic Acid (9)

To a mixture of gabapentin (160 mg, 2.76 mmol) and triethylamine (0.77 mL, 5.5 mmol) in dichloromethane (30 mL) was added trimethylchlorosilane (0.71 mL, 5.5 mmol) and the resulting mixture was stirred until a clear solution was formed. To the above solution was added a solution of α-propanoyloxyisobutyl-p-nitrophenyl carbonate (11) (0.39 g, 1.4 mmol) in dichloromethane (10 mL). After stirring for 30 minutes the reaction mixture was washed with 10% citric acid (20 mL) and the organic layer was separated. The aqueous layer was further extracted with ether (3×10 mL) and the combined organic extracts were dried over MgSO₄. After removing the solvent under reduced pressure, the residue was purified by reverse phase preparative HPLC (acetonitrile, water, 1% formic acid) to afford 190 mg (44%) of the title compound (9). ¹H NMR (CD₃OD, 400 MHz): 0.90 (d, J=6.6 Hz, 3H), 0.91 (d, J=6.6 Hz, 3H), 0.98 (t, J=7.6 Hz, 3H), 1.32-1.58 (m, 10H), 1.83 (m, 1H), 2.18 (s, 2H), 2.28 (q, J=7.6 Hz, 2H), 3.25 (s, 2H), 6.52 (d, J=4.4 Hz, 1H). MS (ESI) m/z 344.34 (M+H)⁺.

### EXAMPLE 4

### Experimental Methods for Measurement of SMVT Transport Activity

1. Transporter Cloning

The complete open reading frame of human SMVT (SLC5A6) was amplified from human cDNA prepared from intestinal mRNA. Gene-specific oligonucleotide primers were designed against Genbank sequences (NM-021095). Amplified PCR products were cloned into a modified version of the mammalian expression vector pcDNA3 (termed pMO) that was engineered to contain the 5′ and 3′ untranslated regions from the Xenopus beta-globin gene. All clones were completely sequenced and tested for function by transient transfection in HEK293 cells. Radiolabeled ³H biotin was used to assess SMVT function (see method below).

2. Xenopus Oocyte Expression and Electrophysiology

cRNA for oocyte expression was prepared by linearization of plasmid cDNA and in vitro transcription using T7 polymerase (Epicentre Ampliscribe kit). Xenopus oocytes were prepared and maintained as previously described (Collins et al., PNAS 13:5456-5460 (1997)) and injected with 10-30 ng RNA. Transport currents were measured 2-6 days later using two-electrode voltage-clamp (Axon Instruments). All experiments were performed using a modified oocyte ringers solution (90 mM NaCl, 2 mM KCl, 1.8 mM CaCl₂, 1 mM MgCl₂, and 10 mM NaHEPES, pH 7.4; in Na⁺-free solutions 9 mM choline chloride was substituted for NaCl). The membrane potential of oocytes was held at −60 mV and current traces acquired using PowerLab software (ADInstruments). Full 7-concentration dose-responses were performed for each compound. Current responses at the highest concentration were normalized to the maximal biotin elicited currents (i.e., at 0.5 mM). Half-maximal concentrations were calculated using non-linear regression curve fitting software (Prism) with the Hill co-efficient fixed to 1. To ensure that currents were specific for the over-expressed transporter, all compounds were tested against uninjected oocytes. Since SMVT requires the presence of sodium ions (Na⁺) for transport, we confirmed that the measured transport was due to SMVT transport by measuring current responses of oocytes to the compounds in a Na⁺-free solution in a control experiment.

3. Construction of Stable Cell Lines and IC₅₀ Measurements

Stable clones of CHOK1 cells were obtained by electroporation, selection in G418, and single cell sorting using FACS (flow-activated cell sorting, Cytomation). Stable clones expressing SMVT were identified by enhanced uptake of radiolabeled substrates. For cell uptake studies, stable CHOK1 clones were seeded into polylysine coated 96-well microtiter plates and grown for 2-3 days. Cells were incubated with experimental solutions (combinations of radiolabeled and unlabeled compounds) for 30 minutes at room temperature, washed four times, and lysed in scintillation solution. Accumulation of radiolabeled molecules was measured in a microtiter scintillation plate reader (Perkin Elmer). Inhibition constants (IC₅₀s) were calculated using curve-fitting software (Prism).

TABLE 1
In vitro transport data for selected compounds on
hSMVT-expressing cells

| | IC₅₀ | Vₘₐₓ as a % of |
|---|---|---|
| COMPOUND | (μM) | Biotin Vₘₐₓ |
| Gabapentin | >500 | 0 |
| (1) | 450 | 21 |
| (6) | 100 | 46 |
| (9) | ND | 19 |

IC₅₀ data from radiolabeled competition assay in SMVT-expressing CHO cells
% Max response (relative to biotin) from transporter-expressing oocytes at a test compound concentration of 0.5 mM

4. Measurement of Uptake by LC/MS/MS

Uptake of unlabeled compounds was measured in cells stably expressing SMVT. Cells were plated at a density of 100,000 cells/well in polylysine coated 96-well microtiter plates and assayed 24-48 hours after plating. Test compounds (0.1 to 3 mM final concentration) were added to a buffered saline solution (HBSS) and 0.1 mL of test solutions were added to each well. Cells were allowed to take up test compounds for 20-60 minutes. Test solutions were aspirated and cells washed 4 times with ice-cold HBSS. Cells were then lysed in a 50% ethanol solution (0.04 mL/well) and sonicated 10 minutes. Following sonication, 0.03 mL of lysate was removed and the concentration of test compounds determined by analytical LC/MS/MS. Transporter specific uptake was determined by comparison with control cells lacking transporter expression or transport in the absence of Na⁺.

### EXAMPLE 5

### Evaluation of the SMVT Substrate Activity of a Biotinylated TAT Peptide

This example examines whether a high molecular weight (i.e., greater than 1500 Da) biotinylated decapeptide disclosed in Ramanathan et al, in Pharm. Res. 2001, 18, 950-956 and J. Controlled Release 2001, 77, 199-212 and Stein et al WO 02/062396 is in fact a substrate for SMVT transport, as has been reported by these authors. The biotinylated decapeptide N-acetyl-D-Lys(ε-biotin)-D-Arg-D-Arg-D-Arg-D-Gln-D-Arg-D-Arg-D-Lys-D-Lys-D-Arg-NH₂ was synthesized as described by Stein et al and purified by reverse-phase HPLC to a high level of chemical purity (>99%, estimated by LC/MS). Using the assay conditions outlined in Example 4 above, both in the presence and absence of 100 mM Na⁺ ions (in Na⁺-free solutions K⁺ or choline was used in place of Na⁺ ions) a concentration of 20 μM of this peptide elicited no specific, sodium-dependent current, indicating that this compound is not a substrate for SMVT.

### EXAMPLE 6

### Standard Methods for Determination of Enzymatic Cleavage of Conjugates in Vitro

The stability of conjugates were evaluated in one or more in vitro systems using a variety of tissue preparations following methods known in the art. Tissues were obtained from commercial sources (e.g., Pel-Freez Biologicals, Rogers, AR, or GenTest Corporation, Woburn, Mass.). Experimental conditions used for the in vitro studies are described in Table 2 below. Each preparation was incubated with test compound at 37° C. for one hour. Aliquots (50 μL) were removed at 0, 30, and 60 min and quenched with 0.1% trifluoroacetic acid in acetonitrile. Samples were then centrifuged and analyzed by LC/MS/MS. Stability of drug conjugates towards specific enzymes (e.g., carboxylesterases, cholinesterases, peptidases, etc.) were also assessed in vitro by incubation with the purified enzyme:

Aminopeptidase Stability. Aminopeptidase 1 (Sigma catalog # A-9934) was diluted in deionised water to a concentration of 856 units/mL. Stability studies were conducted by incubating conjugate (5 μM) with 0.856 units/mL aminopeptidase 1 in 50 mM Tris-HCl buffer at pH 8.0 and 37° C. Concentrations of intact conjugate and released drug were determined at zero time and 60 minutes using LC/MS/MS.

Pancreatin Stability: Stability studies were conducted by incubating conjugate (5 μM) with 1 % (w/v) pancreatin (Sigma, P-1625, from porcine pancreas) in 0.025 M Tris buffer containing 0.5 M NaCl (pH 7.5) at 37° C. for 60 min. The reaction was stopped by addition of 2 volumes of methanol. After centrifugation at 14,000 rpm for 10 min, the supernatant was removed. and analyzed by LC/MS/MS.

Caco-2 Homogenate S9 Stability: Caco-2 cells were grown for 21 days prior to harvesting. Culture medium was removed and cell monolayers were rinsed and scraped off into ice-cold 10 mM sodium phosphate/0.15 M potassium chloride, pH 7.4. Cells were lysed by sonication at 4° C. using a probe sonicator. Lysed cells were then transferred into 1.5 mL centrifuge vials and centrifuged at 9000 g for 20 min at 4° C. The resulting supernatant (Caco-2 cell homogenate S9 fraction) was aliquoted into 0.5 mL vials and stored at −80° C. until used.

For stability studies, conjugate (5 μM) was incubated in Caco-2 homogenate S9 fraction (0.5 mg protein per mL) for 60 min at 37° C. Concentrations of intact conjugate and released drug were determined at zero time and 60 minutes using LC/MS/MS.

Preferred conjugates demonstrate at least 1% cleavage to produce the free drug or an active metabolite thereof within a 60 minute period, as summarized in Table 3.

TABLE 2
Standard Conditions for Conjugate In Vitro Metabolism Studies

| | Substrate | |
|---|---|---|
| Preparation | Concentration | Cofactors |
| Rat Plasma | 2.0 μM | None |
| Human Plasma | 2.0 μM | None |
| Rat Liver S9 | 2.0 μM | NADPH |
| (0.5 mg/mL) | | |
| Human Liver S9 | 2.0 μM | NADPH |
| (0.5 mg/mL) | | |
| Human Intestine | 2.0 μM | NADPH |
| S9 | | |
| (0.5 mg/mL) | | |
| Carboxypeptidase | 2.0 μM | None |
| A | | |
| (10 units/mL) | | |
| Caco-2 | 5.0 μM | None |
| Homogenate | | |
| Pancreatin | 5.0 μM | None |
| Aminopeptidase | 5.0 μM | None |

*NADPH generating system, e.g., 1.3 mM NADP+, 3.3 mM glucose-6-phosphate, 0.4 U/mL glucose-6-phosphate dehydrogenase, 3.3 mM magnesium chloride and 0.95 mg/mL potassium phosphate, pH 7.4.

TABLE 3
% of Drug Released from Conjugates (1), (6) and (9) after 60 min. in
Various Tissue Preparations

| | (1) | (6) | (9) |
|---|---|---|---|
| Rat Plasma | 12 | 23 | 69 |
| Human Plasma | 4 | 0 | 7 |
| Rat Liver S9 | 43 | 52 | 62 |
| (0.5 mg/mL) | | | |
| Human Liver S9 | 51 | 63 | 83 |
| (0.5 mg/mL) | | | |
| Caco-2 S9 | 45 | 55 | 65 |
| Pancreatin | 29 | 28 | 25 |

### EXAMPLE 7

### Uptake of SMVT Substrates Following Oral Administration to Rats

The ability of the SMVT transport mechanism to mediate intestinal absorption of pharmaceutically useful quantities of low molecular weight substrates (i.e. <1,500 Da) was probed in the following dose-escalation study in rats using two natural substrates, sodium pantothenate and biotin:

### Step A: Administration Protocol

Test compounds were administered as an intravenous bolus injection or by oral gavage to groups of four to six adult male Sprague-Dawley rats (weight approx 250 g) as solutions in water or water/ethanol (90:10). The doses ranged between 10 and 200 mg per kg body weight (i.e. 0.04-0.8 mmole/kg). Animals were fasted overnight before the study and for 4 hours post-dosing. Blood samples (1.0 mL) were obtained via a jugular vein cannula at intervals over 24 hours after oral dosing. Blood was processed immediately for plasma and plasma was frozen at −80° C. until analyzed.

### Step B: LC/MS/MS Analysis

Concentrations of test compound in plasma were determined using an API 2000 LC/MS/MS instrument equipped with an Agilent 1100 binary pump and an Agilent autosampler. The column was a Zorbax XDB C8 4.6*150 mm column at room temperature. The mobile phases were 0.1% formic acid (A) and acetonitrile with 0.1% formic acid (B). The gradient condition was: 5% B for 1 min, increasing to 98% B in 3 min and maintained for 2.5 min. A Turbo-IonSpray source was used and compounds were determined using the following MRM fragments: pantothenate 220.06/89.94; biotin 245.1/227.1. The peaks were integrated using Analyst 1.1 quantitation software.

Oral bioavailability (F) was determined by comparison of the area under the concentration versus time curve (AUC) following oral administration of the conjugate or intravenous administration of the parent agent at equimolar doses. Although oral bioavailabilities were found to decrease with increasing dose (biotin: F=100% at 25 mg/kg, F=27% at 200 mg/kg; pantothenate: F=35% at 25 mg/kg, F=10% at 200 mg/kg), significant plasma concentrations of these compounds were observed at the highest doses (i.e. both˜4 μg/mL at 0.82 mmole/kg) despite rapid metabolic clearance. These results demonstrate that pharmaceutically useful quantities of small molecule SMVT substrates can be delivered to the systemic circulation following oral administration.

### EXAMPLE 8

### Uptake of Gabapentin Following Oral Administration of Conjugate (6) to Rats

### Step A: Administration Protocol

The pharmacokinetics of the conjugate (6) was examined in rats. Three groups of four male Sprague-Dawley rats (approx 200 g) with jugular cannulae each received one of the following treatments: A) a single bolus intravenous injection of gabapentin (25 mg/kg, as a solution in water); B) a single oral dose of gabapentin (25 mg/kg, as a solution in water) administered by oral gavage; C) a single oral dose of conjugate (25 mg-equivalents of gabapentin per kg body weight, as a solution in water) administered by oral gavage. Animals were fasted overnight prior to dosing and until 4 hours post-dosing. Serial blood samples were obtained over 24 hours following dosing and blood was processed for plasma by centrifugation. Plasma samples were stored at −80° C. until analyzed. Concentrations of conjugate or gabapentin in plasma samples were determined by LC/MS/MS as described below. Plasma (50 μL) was precipitated by addition of 100 mL of methanol and supernatant was injected directly onto the LC/MS/MS system.

### Step B: Sample Preparation for Absorbed Drug

1. In blank 1.5 mL eppendorf tubes, 300 μL of 50/50 acetonitrile/methanol and 20 μL of p-chlorophenylalanine was added as an internal standard.

2. Rat blood was collected at different time points and immediately 100 μL of blood was added into the eppendorf tube and vortexed to mix.

3. 10 μL of a gabapentin standard solution (0.04, 0.2, 1, 5, 25, 100 μg/mL) was added to 90 μL of blank rat blood to make up a final calibration standard (0.004, 0.02, 0.1, 0.5, 2.5, 10 μg/mL). Then 300 μL of 50/50 acetonitrile/methanol was added into each tube followed by 20 μL of p-chlorophenylalanine.

4. Samples were vortexed and centrifuged at 14,000 rpm for 10 min.

5. Supernatant was taken for LC/MS/MS analysis.

### Step C: LC/MS/MS Analysis

An API 2000 LC/MS/MS spectrometer equipped with Shimadzu 10 ADVp binary pumps and a CTC HTS-PAL autosampler were used in the analysis. A Zorbax XDB C8 4.6 ×150 mm column was heated to 45° C. during the analysis. The mobile phase was 0.1 % formic acid (A) and acetonitrile with 0.1% formic acid (B). The gradient condition was: 5% B for 1 min, then to 98% B in 3 min, then maintained at 98% B for 2.5 min. The mobile phase was returned to 5% B for 2 min. A TurbolonSpray source was used on the API 2000. The analysis was done in positive ion mode and an MRM transition of 172/137 was used in the analysis of gabapentin (MRM transitions 330/198 for (6) were used). 20 μL of the samples were injected. The peaks were integrated using Analyst 1.1 quantitation software. Following oral administration of gabapentin conjugate (6), concentrations of conjugate and gabapentin in plasma were monitored over 24 hours. Oral bioavailability was determined by comparison of area under the gabapentin concentration versus time curve (AUC) following oral administration of conjugate or intravenous administration of an equimolar dose of gabapentin. The oral bioavailability of the conjugate as gabapentin was determined to be >50%, confirming that this conjugate, a good SMVT substrate, was well absorbed in rats after oral dosing.

### EXAMPLE 9

### Uptake of Gabapentin Following Oral Administration of (6) to Monkeys

The pharmacokinetics of the conjugate (6) was examined in cynomolgus monkeys. The conjugate was administered orally to four adult male monkeys (approximate body weight of 6.5 kg) via an oral nasogastric tube as solutions in water. The dose was 10 mg-equivalents of gabapentin per kg body weight. Animals were fasted overnight before the study and for 4 hours post-dosing. Blood samples (1.0 mL) were obtained via femoral or cephalic venipuncture at intervals over 48 hours after oral dosing. Blood was processed immediately for plasma and plasma was frozen at −80° C. until analyzed. Concentrations of (6) or gabapentin in plasma samples were determined by LC/MS/MS as previously described. The oral bioavailability of the conjugate as gabapentin was determined to be >50%, confirming that this conjugate, a good SMVT substrate, was well absorbed in monkeys after oral dosing.

The above examples are illustrative only and do not define the invention; other variants will be readily apparent to those of ordinary skill in the art. The scope of the invention is encompassed by the claims of any patent(s) issuing herefrom. The scope of the invention should, therefore, be determined not with reference to the above description, but instead should be determined with reference to the issued claims along with their full scope of equivalents. Unless otherwise apparent from the context each element, feature, limitation or embodiment of the invention can be used in any combination with one another.

All publications, references, and patent documents cited in this application are incorporated by reference in their entirety for all purposes to the same extent as if each individual publication or patent document were so individually denoted.

1. A method of delivering an agent to a patient, comprising:
orally administering a conjugate to the patient, the conjugate comprising the agent linked to a cleavable conjugate moiety, the conjugate having a molecular weight of less than 1,500 Da and a Vₘₐₓ for the SMVT transporter of at least 5% of the Vₘₐₓ of substrate biotin for the SMVT transporter, wherein the agent without the conjugate moiety, or a metabolite of the agent, has a pharmacological activity and the conjugate has a greater Vₘₐₓ for the SMVT transporter than the agent without the conjugate moiety.

2. The method of claim 1, wherein oral administration of the conjugate provides higher oral agent bioavailability than oral administration of the agent without the conjugate moiety, at equivalent molar doses of the agent and the conjugate.

3. The method of claim 1, wherein the conjugate has a Vₘₐₓ for the SMVT transporter of at least 10% of the Vₘₐₓ of substrate biotin for the SMVT transporter.

4. The method of claim 1, wherein the conjugate has a Vₘₐₓ for the SMVT transporter of at least 20% of the Vₘₐₓ of substrate biotin for the SMVT transporter.

5. The method of claim 1, wherein the conjugate has a Vₘₐₓ for the SMVT transporter of at least 50% of the Vₘₐₓ of substrate biotin for the SMVT transporter.

6. The method of claim 1, wherein the conjugate has a Vₘₐₓ for the SMVT transporter of at least 100% of the Vₘₐₓ of substrate biotin for the SMVT transporter.

7. The method of claim 1, wherein the conjugate or the conjugate moiety has been identified by screening a plurality of candidate substrates for transport through the SMVT transporter.

8. The method of claim 1, wherein the conjugate or the conjugate moiety has been identified by screening a plurality of candidate substrates for transport through the SMVT transporter and another transporter.

9. The method of claim 8, wherein the plurality of candidate substrates were screened separately for transport through the SMVT transporter and the other transporter.

10. The method of claim 1, further comprising screening a plurality of conjugates or conjugate moieties to identify the conjugate or conjugate moiety.

11. A conjugate comprising an agent cleavably linked to a conjugate moiety, the conjugate being a substrate for an SMVT transporter, the conjugate having a molecular weight of less than 1,500 Da and a Vₘₐₓ of at least 5% of biotin for the SMVT transporter, wherein the agent without the conjugate moiety, or a metabolite of the agent, has a pharmacological activity, and the conjugate has a greater Vₘₐₓ for the SMVT transporter than the agent without the conjugate moiety.

12. The conjugate of claim 11, formulated with a carrier for oral delivery.

13. The conjugate of claim 11, wherein the conjugate is not pharmacologically active.

14. The conjugate of claim 13, formulated with a carrier for oral delivery.

15. The conjugate of claim 11 or 13, wherein oral administration of the conjugate provides higher oral agent bioavailability than oral administration of the agent without the conjugate moiety, at equivalent molar doses of the agent and the conjugate.

16. The conjugate of claim 11 or 13, wherein the conjugate has a Vₘₐₓ for the SMVT transporter of at least 10% of the Vₘₐₓ of substrate biotin for the SMVT transporter.

17. The conjugate of claim 11 or 13, wherein the conjugate has a Vₘₐₓ for the SMVT transporter of at least 20% of the Vₘₐₓ of substrate biotin for the SMVT transporter.

18. The conjugate of claim 11 or 13, wherein the conjugate has a Vₘₐₓ for the SMVT transporter of at least 50% of the Vₘₐₓ of substrate biotin for the SMVT transporter.

19. The conjugate of claim 11 or 13, wherein the conjugate has a Vₘₐₓ for the SMVT transporter of at least 100% of the Vₘₐₓ of substrate biotin for the SMVT transporter.

20. The conjugate of claim 11 or 13, where the agent without the conjugate moiety has a Vₘₐₓ for the SMVT transporter of less than 5% of the Vₘₐₓ of substrate biotin for the SMVT transporter.

21. The conjugate of claim 11 or 13, wherein the linker comprises a covalent bond that is cleavable in vivo.

22. The conjugate of claim 11 or 13, wherein the agent and the conjugate moiety are linked via a peptide.

23. The conjugate of claim 12 or 14, wherein the carrier comprises an immediate release formulation.

24. The conjugate of claim 12 or 14, wherein the carrier comprises a sustained release formulation.

25. The conjugate of claim 11 or 13, wherein the agent has no carboxylic acid functionality.

26. The conjugate of claim 11 or 13, wherein the conjugate moiety contains a carboxylic acid funtionality.

27. A method of screening a conjugate for transport by an SMVT transporter, comprising:
providing a conjugate comprised of an agent linked to a conjugate moiety, the conjugate having a molecular weight of less than 1500 Da;
providing a cell expressing an SMVT transporter;
contacting the cell with the conjugate; and
determining whether the conjugate passes into and/or through the cell by way of the transporter.

28. The method of claim 27, wherein the cell is transfected with DNA encoding the SMVT transporter.

29. The method of claim 27, wherein the cell is an oocyte injected with nucleic acid encoding the SMVT transporter.

30. The method of claim 27, comprising:
providing a second cell expressing another transporter and lacking an SMVT transporter;
contacting the second cell with the conjugate; and
determining whether the conjugate passes through the transporter.

31. The method of claim 30, wherein the conjugate is contacted with the second cell before being contacted with the first cell.

32. A method of making a pharmaceutical composition, comprising
linking an agent to a conjugate moiety to form a conjugate, the conjugate having a molecular weight of less than 1,500 Da, wherein the conjugate is transported by the SMVT transporter with a Vₘₐₓ of at least 5% of the Vₘₐₓ of substrate biotin; and
formulating the conjugate with a carrier as a pharmaceutical composition.

33. The method of claim 32, wherein the conjugate exhibits higher oral agent bioavailability than the agent without the conjugate moiety, at equivalent molar doses of the agent and the conjugate.

34. The method of claim 32, wherein the conjugate has a Vₘₐₓ for the SMVT transporter of at least 10% of the Vₘₐₓ of substrate biotin for the SMVT transporter.

35. The method of claim 32, wherein the conjugate has a Vₘₐₓ for the SMVT transporter of at least 20% of the Vₘₐₓ of substrate biotin for the SMVT transporter.

36. The method of claim 32, wherein the conjugate has a Vₘₐₓ for the SMVT transporter of at least 50% of the Vₘₐₓ of substrate biotin for the SMVT transporter.

37. The method of claim 32, wherein the conjugate has a Vₘₐₓ for the SMVT transporter of at least 100% of the Vₘₐₓ of substrate biotin for the SMVT transporter.

38. The method of claim 32, comprising screening the conjugate moiety for capacity to mediate transport via the SMVT transporter.

39. A method of making a pharmaceutical product, comprising:
forming a plurality of different conjugates, each conjugate comprising a single therapeutic agent linked to one of a plurality of different cleavable conjugate moieties, the agent without any conjugate moiety, or an active metabolite of the agent, having a pharmacological activity, each of the conjugates having a molecular weight of less than 1,500 Da;
screening the plurality of conjugates for SMVT transport;
selecting a conjugate having a Vₘₐₓ for the SMVT transporter of at least 5% of the Vₘₐₓ of substrate biotin for the SMVT transporter, the selected conjugate having a greater Vₘₐₓ for the SMVT transporter than the agent without the selected conjugate moiety; and
formulating the selected conjugate as a pharmaceutical product.
